(19) United States
(12) Patent Application Publication
(10) Pub. No.: US 2026/0290561 A1
(43) Pub. Date: Sep. 24, 2026

(54) METHOD AND SYSTEM FOR AUTOMATED PATIENT COORDINATION

(71) Applicant: Qure.ai Technologies Private Limited, Mumbai (IN)

(72) Inventors: Bhargava Reddy, Mumbai (IN); Preetham Putha, Mumbai (IN); Rohitashva Agrawal, Mumbai (IN); Manoj Tadepalli, Mumbai (IN); Polamoni Ranjana Devi, Mumbai (IN); Sai Vinayak Mukkavilli, Mumbai (IN)

(21) Appl. No.: 19/449,477

(22) Filed: Jan. 15, 2026

(30) Foreign Application Priority Data
Mar. 18, 2025 (IN) 202521024266

(51) Int. Cl. G16H 40/20; G06T 7/00; G16H 15/00

(52) U.S. Cl. CPC G16H 40/20; G06T 7/0012; G16H 15/00; G06T 2200/24; G06T 2207/10081; G06T 2207/10088; G06T 2207/10104; G06T 2207/10116; G06T 2207/10132; G06T 2207/20081; G06T 2207/20084

(57) ABSTRACT

The system and method for automated patient coordination comprises a processor (202) and memory (204) configured to execute instructions for optimizing automated patient coordination. The system (100) receives one or more medical images associated with users and displays corresponding worklists to stakeholders based on these images. Further, the system (100) provides a communication interface for stakeholders to collaborate in real-time based on the one or more worklists. Further, the one or more worklists and the communication interface are integrated with an image viewing interface to facilitate efficient viewing and discussion of medical images. Additionally, the system (100) generates a patient coordination report based on findings from the worklists and collaborative communications between stakeholders.

### CROSS-REFERENCE TO RELATED APPLICATIONS AND PRIORITY

The present application claim priority from Indian Patent Application No. 202521024266 filed on Mar. 18, 2025.

### TECHNICAL FIELD

The presently disclosed embodiments are related, in general, to the field of patient coordination. More particularly, the presently disclosed embodiments are related to a method and system for automated patient coordination.

### BACKGROUND

This section is intended to introduce the reader to various aspects of art (the relevant technical field or area of knowledge to which the invention pertains), which may be related to various aspects of the present disclosure that are described or claimed below. This discussion is believed to be helpful in providing the reader with background information to facilitate a better understanding of the various aspects of the present disclosure. Accordingly, it should be understood that these statements in this background section are to be read in this light, and not as admissions of prior art. Similarly, a problem mentioned in the background section.

The healthcare sector is continuously striving to improve patient management, diagnosis, and treatment coordination, driven by the increasing complexity of medical care and the growing demands of an aging population. As medical knowledge and technologies evolve, healthcare professionals face the challenge of managing large volumes of patient data from a variety of sources, such as imaging, lab results, and electronic health records (EHRs), while also needing to make fast, accurate clinical decisions. This growing complexity, coupled with the need for timely and accurate diagnosis and treatment, has led to a push for more integrated, streamlined healthcare systems. Improving patient management is not only about providing care but also about ensuring that care is efficient, coordinated, and patient centred.

However, current healthcare workflows remain highly fragmented, with clinicians relying on a variety of separate platforms and tools. These include PACS (Picture Archiving and Communication Systems) for imaging, AI software for diagnostic assistance, reporting platforms for generating medical documentation, and communication tools such as WhatsApp or email for coordinating patient care. This fragmented system poses numerous challenges, making patient management cumbersome and increasing the risk of errors, delays, and inefficiencies. The invention proposes a unified platform that integrates all these functionalities into a single system, eliminating the need to switch between multiple platforms, and enhancing both clinician workflow and patient outcomes.

The primary issue with existing technology is fragmentation. Clinicians are required to log in to various standalone systems, each designed to handle a specific task, such as imaging, AI-based detection, reporting, and communication. This approach creates several inefficiencies: clinicians spend valuable time switching between different systems, valuable patient data is often spread across various platforms, and there is a higher likelihood of human error as data must be manually transferred or re-entered into multiple systems. These workflows also create significant delays in decision-making, as clinicians must access different platforms to gather the full scope of patient information. This delay can be critical, particularly in urgent cases, and ultimately affects patient care and outcomes.

Traditional AI-based disease detection systems are one of the most widely used tools in healthcare today. These standalone AI systems are designed to detect abnormalities such as stroke, lung nodules, and intracranial haemorrhages from imaging studies like CT scans or X-rays. While these AI tools offer powerful detection capabilities through deep learning algorithms, they are typically isolated from the rest of the clinical workflow. Clinicians often face difficulties accessing AI-generated findings because they are not integrated with other systems like PACS, leading to delays or even missed insights. Furthermore, AI outputs are often not automatically included in medical reports, which requires additional manual entry, increasing the risk of human error and miscommunication.

Quantification and reporting are another key area in which existing tools often fall short. Some reporting platforms generate structured reports based on clinician inputs, and some even integrate AI-generated findings. However, these reporting systems are frequently disjointed from imaging and AI software, forcing clinicians to switch between multiple tools to create comprehensive reports. Additionally, manual transcription errors are common, as AI findings are often not directly linked to reports, potentially leading to missing or misinterpreted findings. Compatibility issues also arise, as many reporting tools lack HL7/DICOM integration with hospital EHR (Electronic Health Records) or PACS systems, complicating the sharing of medical data and slowing down the process of updating patient records.

Worklist management systems, often part of PACS, are another area where traditional methods fail to provide an optimal solution. While PACS offers basic worklist sorting based on time, patient ID, or modality, these systems do not prioritize cases based on their severity or urgency. Critical cases such as stroke may go unnoticed if they are buried in a long list of routine cases. Additionally, PACS worklists are usually siloed, meaning that radiologists, neurologists, and emergency room physicians might each have separate worklists. This lack of cross-departmental coordination can lead to delays in decision-making, and clinicians may need to manually sort cases, which wastes valuable time and can delay urgent treatment.

In addition, the lack of comprehensive integration between imaging data, AI insights, and clinical workflows makes it difficult to provide a cohesive, personalized approach to patient management, also to provide health status based on multiple pathologies detected for the same person. Further, the traditional worklist management system does not automatically highlight critical cases, resulting in missed diagnoses. Clinicians must manually filter and prioritize cases manually. As a result, clinicians are often forced to follow standardized workflows that do not align with their clinical judgment or the needs of individual patients. This reduces efficiency, increases the likelihood of errors, and can delay critical care. The fragmented nature of these systems increases complexity, wastes time, and heightens the risk of human error as clinicians are required to manually transfer data between platforms and log into multiple systems.

Emergency alerts and notifications are essential for timely diagnosis and intervention, particularly in cases of acute medical conditions like strokes or intracranial haemorrhages. Standalone AI tools can detect these critical findings, but the existing notification systems often fail to alert all relevant healthcare providers in real-time, which leads to delayed responses. In addition, alerts are typically sent through informal channels like email, text messages or instant messages, which are disconnected from the patient’s imaging or medical records. Clinicians must manually access separate systems to review the images or reports associated with the alert, adding unnecessary steps and delays to the response process. Furthermore, current alert systems may lack customization, meaning clinicians cannot tailor alerts to suit the severity or urgency of different conditions.

Communication between clinicians is another critical challenge. Many healthcare professionals use external messaging apps or email to coordinate care, but these platforms are not designed for medical workflows. They lack integration with medical systems, meaning that important clinical data such as imaging findings, AI results, or patient reports are not easily shared within these communication tools. Moreover, using unsecured messaging apps exposes patient data to privacy and security risks, violating HIPAA and GDPR regulations. The lack of patient-centric conversation threads further complicates collaboration, making it difficult for clinicians to track important discussions and decisions about a patient’s care.

The traditional health management systems often lack the ability to automate the detection of pathological features, integrate clinical and imaging data seamlessly, or offer real-time communication and collaboration capabilities.

In view of the above, addressing the aforementioned technical challenges requires an improved method and a system for automated patient coordination.

Further limitations and disadvantages of conventional and traditional approaches will become apparent to one of skill in the art, through comparison of described systems with some aspects of the present disclosure, as set forth in the remainder of the present application and with reference to the drawings.

### SUMMARY

This summary is provided to introduce concepts related to a method and a system for automated patient coordination and the concepts are further described below in the detailed description. This summary is not intended to identify essential features of the claimed subject matter nor is it intended for use in determining or limiting the scope of the claimed subject matter.

According to embodiments illustrated herein, the method for automated patient coordination is disclosed. The method may be implemented by an application server including one or more processors and a memory communicatively coupled to the one or more processors and the memory is configured to store processor executable programmed instructions. Further, the method may comprise a step of receiving one or more medical images associated with one or more users. Further, the method may comprise a step of displaying one or more worklists to one or more stakeholders based on the one or more medical images that may be associated with the one or more users. Further, the method may comprise a step of providing a communication interface for collaborative communication between the one or more stakeholders based on the displayed one or more worklists. Further, the method may comprise a step of integrating the one or more worklists and the communication interface with an image viewing interface for facilitating viewing of the one or more medical images that may be based on the collaborative communication. Furthermore, the method may comprise a step of providing a patient coordination report based on findings from the one or more worklists and the collaborative communication between the one or more stakeholders.

According to embodiments illustrated herein, the system of automated patient coordination is disclosed. Further, the system may comprise a processor and the memory. Further, the memory may be configured to store programmed instructions that cause the processor to perform following operations. Further, the processor may be configured for receiving the one or more medical images associated with the one or more users. Further, the processor may be configured for displaying the one or more worklists to the one or more stakeholders based on the one or more medical images associated with the one or more users. Further, the processor may be configured for providing the communication interface for collaborative communication between the one or more stakeholders based on the displayed one or more worklists. Further, the processor may be configured for integrating the one or more worklists and the communication interface with the image viewing interface for facilitating viewing of the one or more medical images based on the collaborative communication. Furthermore, the processor may be configured for providing the patient coordination report based on findings from the one or more worklists and the collaborative communication between the one or more stakeholders.

According to embodiments illustrated herein, a non-transitory computer-readable storage medium having stored thereon, a set of computer-executable instructions causing a computer comprising the one or more processors to perform steps. Further, the steps may comprise of receiving the one or more medical images associated with the one or more users. Further, the steps may comprise of displaying the one or more worklists to the one or more stakeholders based on the one or more medical images that may be associated with the one or more users. Further, the steps may comprise of providing the communication interface for collaborative communication between the one or more stakeholders based on the displayed one or more worklists. Further, the steps may comprise of integrating the one or more worklists and the communication interface with the image viewing interface for facilitating viewing of the one or more medical images that may be based on the collaborative communication. Furthermore, the steps may comprise of providing the patient coordination report based on findings from the one or more worklists and the collaborative communication between the one or more stakeholders.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF DRAWINGS

The accompanying drawings illustrate the various embodiments of systems, methods, and other aspects of the disclosure. Any person with ordinary skills in art will appreciate that the illustrated element boundaries (e.g., boxes, groups of boxes, or other shapes) in the figures represent one example of the boundaries. In some examples, one element may be designed as multiple elements, or multiple elements may be designed as one element. In some examples, an element shown as an internal component of one element may be implemented as an external component in another, and vice versa. Further, the elements may not be drawn to scale.

Various embodiments will hereinafter be described in accordance with the appended drawings, which are provided to illustrate and not to limit the scope in any manner, wherein similar designations denote similar elements, and in which:

FIG. 1 is a block diagram that illustrates a system (100) for automated patient coordination, in accordance with an embodiment of present subject matter.

FIG. 2 is a block diagram that illustrates various components of an application server (104) configured for automated patient coordination, in accordance with an embodiment of the present subject matter.

FIG. 3 is a flowchart that illustrates a method (300) for automated patient coordination, in accordance with an embodiment of the present subject matter.

FIG. 4 is a flowchart that illustrates a flow (400) of automated patient coordination, in accordance with an exemplary embodiment of present subject matter; and

FIG. 5 illustrates a block diagram (500) of an exemplary computer system for implementing embodiments consistent with the present subject matter.

### DETAILED DESCRIPTION

The present disclosure may be best understood with reference to the detailed figures and description set forth herein. Various embodiments are discussed below with reference to the figures. However, those skilled in the art will readily appreciate that the detailed descriptions given herein with respect to the figures are simply for explanatory purposes as the methods and systems may extend beyond the described embodiments. For example, the teachings presented, and the needs of a particular application may yield multiple alternative and suitable approaches to implement the functionality of any detail described herein. Therefore, any approach may extend beyond the particular implementation choices in the following embodiments described and shown.

References to “one embodiment,” “at least one embodiment,” “an embodiment,” “one example,” “an example,” “for example,” and so on indicate that the embodiment(s) or example(s) may include a particular feature, structure, characteristic, property, element, or limitation but that not every embodiment or example necessarily includes that particular feature, structure, characteristic, property, element, or limitation. Further, repeated use of the phrase “in an embodiment” does not necessarily refer to the same embodiment. The terms “comprise”, “comprising”, “include(s)”, or any other variations thereof, are intended to cover a non-exclusive inclusion, such that a setup, system or method that comprises a list of components or steps does not include only those components or steps but may include other components or steps not expressly listed or inherent to such setup or system or method. In other words, one or more elements in a system or apparatus preceded by “comprises… a” does not, without more constraints, preclude the existence of other elements or additional elements in the system or apparatus.

An objective of the present disclosure is to automate patient coordination by integrating medical images and worklists to provide a seamless experience for stakeholders.

Another objective of the present disclosure is to facilitate efficient communication between stakeholders involved in patient care by providing a dedicated communication interface.

Yet another objective of the present disclosure is to allow stakeholders to access and manage worklists based on relevant medical images.

Yet another objective of the present disclosure is to enhance the workflow by integrating worklists and communication interfaces with an image viewing interface.

Yet another objective of the present disclosure is to improve patient care by providing a patient coordination report that summarizes findings from worklists and collaborative communications.

Yet another objective of the present disclosure is to enable real-time updates and modifications to worklists based on collaborative communication, ensuring that stakeholders are always working with the most current and relevant information available.

Yet another objective of the present disclosure is to enhance the coordination of care by supporting the inclusion of multiple stakeholders with different roles and responsibilities, such as physicians, nurses, and administrative personnel, all of whom can contribute to the decision-making process.

Yet another objective of the present disclosure is to reduce the risk of errors and miscommunication in the coordination of patient care by providing an integrated platform for medical image viewing, worklist management, and stakeholder communication. This minimizes the reliance on manual processes and increases the overall accuracy of patient care coordination.

Yet another objective of the present disclosure is to optimize the overall healthcare workflow by streamlining the exchange of information and ensuring that all necessary steps in patient care coordination are carried out efficiently, reducing delays and improving patient satisfaction.

Yet another objective of the present disclosure is to provide a user-friendly and secure platform to all the patients and healthcare professionals.

The present solution discloses an automated method and a system for patient coordination, focusing on enhancing collaborative communication among healthcare stakeholders using medical images and worklists. The method involves receiving one or more medical images associated with users and displaying corresponding worklists to stakeholders, based on the images. A communication interface is provided for seamless collaboration on the displayed worklists, facilitating efficient coordination. This solution integrates the worklists and communication interface with an image viewing interface to support real-time interaction and decision-making. Additionally, the method includes generating a patient coordination report that consolidates findings from the worklists and communication among stakeholders. The solution further improves coordination by automatically detecting pathological features from the medical images using advanced deep learning techniques, identifying conditions such as infectious, neurological, and oncological diseases. The worklists are dynamically displayed, prioritizing critical conditions, and allowing stakeholders to filter, sort, and update information in real-time. The method also integrates clinical data, such as medical history and risk factors, with the images to enhance the accuracy of pathological feature detection and classification. This comprehensive approach improves collaboration, timely decision-making, and ensures optimized patient care through integration.

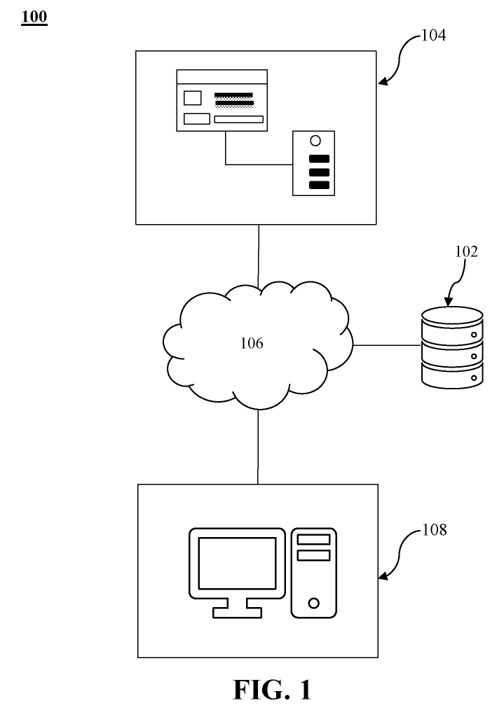

FIG. 1 is a block diagram that illustrates an automated patient coordination system (100), in accordance with an embodiment of present subject matter. The system (100) typically includes a database server (102), an application server (104), a communication network (106), and one or more portable devices (108). The database server (102), the application server (104), and the one or more portable devices (108) are typically communicatively coupled with each other via the communication network (106). In an embodiment, the application server (104) may communicate with the database server (102), and the one or more portable devices (108) using one or more protocols such as, but not limited to, Hypertext Transfer Protocol (HTTP), Transmission Control Protocol/Internet Protocol (TCP/IP), Wireless Application Protocol (WAP), RF mesh, Bluetooth Low Energy (BLE), and the like, to communicate with one another.

In one embodiment, the database server (102) may refer to a computing device configured to store one or more medical images associated with users, one or more worklists to one or more stakeholders based on the one or more medical images associated with the one or mor users. Further, the database server (102) may be configured to communicate with the communication interface for collaborative communication between the one or more stakeholders based on the displayed one or more worklists. Further, the database server (102) may be configured to store patient’s one or more clinical parameters, one or more non-clinical parameters, and a combination thereof.

In an embodiment, the database server (102) may include a special purpose operating system specifically configured to perform one or more database operations on the stored content. Examples of database operations may include, but are not limited to, Select, Insert, Update, and Delete. In an embodiment, the database server (102) may include hardware that may be configured to perform one or more predetermined operations. In an embodiment, the database server (102) may be realized through various technologies such as, but not limited to, Microsoft® SQL Server, Oracle®, IBM DB2®, Microsoft Access®, PostgreSQL®, MySQL®, SQLite®, distributed database technology and the like. In an embodiment, the database server (102) may be configured to utilize the application server (104) for automated patient coordination.

A person with ordinary skills in art will understand that the scope of the disclosure is not limited to the database server (102) as a separate entity. In an embodiment, the functionalities of the database server (102) can be integrated into the application server (104) or into the one or more portable device (108).

In an embodiment, the application server (104) may refer to a computing device or a software framework hosting an application or a software service. In an embodiment, the application server (104) may be implemented to execute procedures such as, but not limited to, programs, routines, or scripts stored in one or more memories for supporting the hosted application or the software service. In an embodiment, the hosted application or the software service may be configured to perform one or more predetermined operations. The application server (104) may be realized through various types of application servers such as, but are not limited to, a Java application server, a .NET framework application server, a Base4 application server, a PHP framework application server, or any other application server framework.

In an embodiment, the application server (104) may be configured to utilize the database server (102) and the one or more portable devices (108), in conjunction, for implementing the method for automated patient coordination. In an implementation, the application (104) serves as the infrastructure for executing the method for automated patient coordination. The application server (104) may be configured to receive one or more medical images associated with one or more users. Further, the application server (104) may be configured to display one or more stakeholders based on the one or more medical images associated with the one or more users. Further, the application server (104) may be configured to provide a communication interface for collaborative communication between the one or more stakeholders based on the displayed one or more worklists. Further, the application server (104) may be configured to integrate the one or more worklists and the communication interface with an image viewing interface for facilitating viewing of the one or more medical images based on the collaborative communication. Further, the application server (104) may be configured to provide a patient coordination report based on findings from the one or more worklists and the collaborative communication between the one or more stakeholders.

In one embodiment, the application server (104) may be configured to automatically detect the one or more pathological features from the one or more medical images. Further, the application server (104) may be configured to generate a real-time notification alert based on detecting the one or more pathological features belonging to one or more critical conditions.

In an embodiment, the communication network (106) may correspond to a communication medium through which the application server (104), the database server (102), and the one or more portable device (108) may communicate with each other. Such a communication may be performed in accordance with various wired and wireless communication protocols. Examples of such wired and wireless communication protocols include, but are not limited to, Transmission Control Protocol and Internet Protocol (TCP/IP), User Datagram Protocol (UDP), Hypertext Transfer Protocol (HTTP), Wireless Application Protocol (WAP), File Transfer Protocol (FTP), ZigBee, EDGE, infrared IR), IEEE 802.11, 802.16, 2G, 3G, 4G, 5G, 6G, 7G cellular communication protocols, and/or Bluetooth (BT) communication protocols. The communication network (106) may either be a dedicated network or a shared network. Further, the communication network (106) may include a variety of network devices, including routers, bridges, servers, computing devices, storage devices, and the like. The communication network (106) may include, but is not limited to, the Internet, intranet, a cloud network, a Wireless Fidelity (Wi-Fi) network, a Wireless Local Area Network (WLAN), a Local Area Network (LAN), a cable network, the wireless network, a telephone network (e.g., Analog, Digital, POTS, PSTN, ISDN, xDSL), a telephone line (POTS), a Metropolitan Area Network (MAN), an electronic positioning network, an X.25 network, an optical network (e.g., PON), a satellite network (e.g., VSAT), a packet-switched network, a circuit-switched network, a public network, a private network, and/or other wired or wireless communications network configured to carry data.

In an embodiment, the one or more portable devices (108) may refer to a computing device used by a user. The one or more portable devices (108) may comprise of one or more processors and one or more memory. The one or more memories may include computer readable code that may be executable by one or more processors to perform predetermined operations. In an embodiment, the one or more portable devices (108) may present a web user interface for the automated patient coordination using the application server (104). Example web user interfaces presented on the one or more portable devices (108) and relevant information, for the automated patient coordination. Examples of the one or more portable devices (108) may include, but are not limited to, a personal computer, a laptop, a computer desktop, a personal digital assistant (PDA), a mobile device, a tablet, or any other computing device.

The system (100) can be implemented using hardware, software, or a combination of both, which includes using where suitable, one or more computer programs, mobile applications, or “apps” by deploying either on-premises over the corresponding computing terminals or virtually over cloud infrastructure. The system (100) may include various micro-services or groups of independent computer programs which can act independently in collaboration with other micro-services. The system (100) may also interact with a third-party or external computer system. Internally, the system (100) may be the central processor of all requests for transactions by the various actors or users of the system.

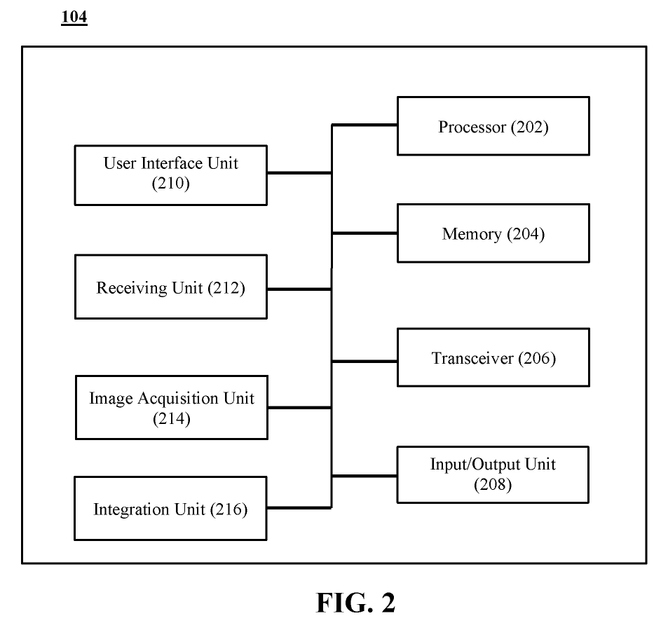

FIG. 2 illustrates a diagram illustrating various components of the application server (104) configured for automated patient coordination, in accordance with an embodiment of the present subject matter. Further, FIG. 2 is explained in conjunction with elements from FIG. 1. Here, the application server (104) preferably includes a processor (202), a memory (204), a transceiver (206), an Input/Output unit (208), a user interface unit (210), a receiving unit (212), an image acquisition unit (214), and an integration unit (216). The processor (202) is further preferably communicatively coupled to the memory (204), the transceiver (206), the Input/Output unit (208), the user interface unit (210), the receiving unit (212), the image acquisition unit (214), and the integration unit (216), while the transceiver (206) is preferably communicatively coupled to the communication network (106).

The processor (202) comprises suitable logic, circuitry, interfaces, and/or code that may be configured to execute a set of instructions stored in the memory (204), and may be implemented based on several processor technologies known in the art. The processor (202) works in coordination with the transceiver (206), the Input/Output unit (208), the user interface unit (210), the receiving unit (212), the image acquisition unit (214), and the integration unit (216) for automated patient coordination. Examples of the processor (202) include, but not limited to, standard microprocessor, microcontroller, central processing unit (CPU), an X86-based processor, a Reduced Instruction Set Computing (RISC) processor, an Application- Specific Integrated Circuit (ASIC) processor, and a Complex Instruction Set Computing (CISC) processor, distributed or cloud processing unit, state machines, logic circuitries, and/or any devices that manipulate signals based on operational instructions and/or other processing logic that accommodates the requirements of the present invention.

The memory (204) comprises suitable logic, circuitry, interfaces, and/or code that may be configured to store the set of instructions, which are executed by the processor (202). Preferably, the memory (204) is configured to store one or more programs, routines, or scripts that are executed in coordination with the processor (202). Additionally, the memory (204) may include any computer-readable medium or computer program product known in the art including, for example, volatile memory, such as static random-access memory (SRAM) and dynamic random-access memory (DRAM), and/or non-volatile memory, such as read only memory (ROM), erasable programmable ROM, a Hard Disk Drive (HDD), flash memories, Secure Digital (SD) card, Solid State Disks (SSD), optical disks, magnetic tapes, memory cards, virtual memory and distributed cloud storage. The memory (204) may be removable, non-removable, or a combination thereof. Further, the memory (204) may include routines, programs, objects, components, data structures, etc., which perform particular tasks or implement particular abstract data types. The memory (204) may include programs or coded instructions that supplement applications and functions of the system (100). In one embodiment, the memory (204), amongst other things, serves as a repository for storing data processed, received, and generated by one or more of the programs or the coded instructions. In yet another embodiment, the memory (204) may be managed under a federated structure that enables adaptability and responsiveness of the application server (104).

The transceiver (206) comprises suitable logic, circuitry, interfaces, and/or code that may be configured to receive, process or transmit information, data or signals, which are stored by the memory (204) and executed by the processor (202). The transceiver (206) is preferably configured to receive, process or transmit, one or more programs, routines, or scripts that are executed in coordination with the processor (202). The transceiver (206) is preferably communicatively coupled to the communication network (106) of the system (100) for communicating all the information, data, signal, programs, routines or scripts through the network. The transceiver (206) may be configured to receive a communication request.

The transceiver (206) may implement one or more known technologies to support wired or wireless communication with the communication network (106). In an embodiment, the transceiver (206) may include, but is not limited to, an antenna, a radio frequency (RF) transceiver, one or more amplifiers, a tuner, one or more oscillators, a digital signal processor, a Universal Serial Bus (USB) device, a coder-decoder (CODEC) chipset, a subscriber identity module (SIM) card, and/or a local buffer. Also, the transceiver (206) may communicate via wireless communication with networks, such as the Internet, an Intranet and/or a wireless network, such as a cellular telephone network, a wireless local area network (LAN) and/or a metropolitan area network (MAN). Accordingly, the wireless communication may use any of a plurality of communication standards, protocols and technologies, such as: Global System for Mobile Communications (GSM), Enhanced Data GSM Environment (EDGE), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11b, IEEE 802.11g and/or IEEE 802.11n), voice over Internet Protocol (VoIP), Wi-MAX, a protocol for email, instant messaging, and/or Short Message Service (SMS).

The input/output (I/O) unit (208) comprises suitable logic, circuitry, interfaces, and/or code that may be configured to receive or present information. The input/output unit (208) comprises various input and output devices that are configured to communicate with the processor (202). Examples of the input devices include, but are not limited to, a keyboard, a mouse, a joystick, a touch screen, a microphone, a camera, and/or a docking station. Examples of the output devices include, but are not limited to, a display screen and/or a speaker. The I/O unit (208) may include a variety of software and hardware interfaces, for example, a web interface, a graphical user interface, and the like. The I/O unit (208) may allow the system (100) to interact with the user directly or through the portable devices (108). Further, the I/O unit (208) may enable the system (100) to communicate with other computing devices, such as web servers and external data servers (not shown). The I/O unit (208) can facilitate multiple communications within a wide variety of networks and protocol types, including wired networks, for example, LAN, cable, etc., and wireless networks, such as WLAN, cellular, or satellite. The I/O unit (208) may include one or more ports for connecting a number of devices to one another or to another server. In one embodiment, the I/O unit (208) allows the application server (104) to be logically coupled to other portable devices (108), some of which may be built in. Illustrative components include tablets, mobile phones, desktop computers, wireless devices, etc.

In an embodiment, the I/O unit (208) may be configured to provide a patient report based on findings from the one or more worklists and the collaborative communication between the one or more stakeholders. Further, the I/O unit (208) may be configured to generate a real-time notification alert based on detecting the one or more pathological features that may belong to the one or more pathological features belonging to one or more critical conditions. Further, the one or more critical conditions may include, but are not limited to, Fractures, Stroke, Pneumothorax, bleeds, Intracranial Haemorrhage (ICH), Acute Stroke, Pulmonary Embolism, Myocardial Infarction, Aortic Dissection, Sepsis, Brain Tumors, Large Vessel Occlusion (LVO), acute respiratory distress syndrome (ARDS), life-threatening arrhythmias, midline shift, stroke, lung nodule findings. These alerts ensure that clinicians and healthcare teams are immediately notified of the presence of potentially life-threatening conditions, enabling timely intervention and improving patient outcomes. Further, the worklist containing the one or more pathological features belonging to the one or more critical conditions, is automatically moved to top of the one or more worklists displayed to the one or more stakeholders.

Further, the I/O unit (208) may display the patient coordination report on the image viewing interface. Further, the patient coordination report may be generated by consolidating the findings from the one or more worklists and the collaborative communication between the one or more stakeholders. Further, the findings in the patient coordination report may be updated by the one or more stakeholders. For example, the one or more stakeholders, if required, can provide additional notes on the findings generated in the corresponding worklists for a particular user. Further, the one or more pathological features in the one or more worklists may be updated by the one or more stakeholders.

In one embodiment, the user interface unit (210) of the application server (104) is disclosed. The user interface unit (210) comprises suitable logic, circuitry, interfaces, and/or code that may be configured for automated patient coordination. Further, the user interface unit (210) may be configured to display the one or more worklists to the one or more stakeholders on the one or more medical images that may be associated with the one or more users. Further, the one or more stakeholders may be a radiologist, health care professional, hospital admin, or a combination thereof. Further, displaying of the one or more worklists may be performed based on the detection of the one or more pathological features from the one or more medical images. Further, the each of the one or more worklists comprises at least one or more clinical parameters, one or more non-clinical parameters, or a combination thereof. Further, the one or more clinical parameters comprises at least one of the one or more pathological features, type of disease, report status, finding present, finding absent or a combination thereof. Further, the one or more non-clinical parameters comprises at least one of report status, urgency, physician preference, sorting and filtering the one or more worklists. Further, the sorting and searching may be based on dates, sites, status, but not limited to finding present, finding absent, pending actions, modalities, report status, patient demographics, or a combination thereof.

Further, the user interface unit (210) may be configured to provide one or more functionalities on the one or more worklists based on one of the one or more clinical parameters, the one or more non-clinical parameters, or a combination thereof. Further, the one or more functionalities may correspond to one of filtering the one or more worklists displayed based on selecting one of the one or more clinical parameters, the one or more non-clinical parameters, or a combination thereof by the one or more stakeholders. Further, the one or more functionalities may correspond to sorting the one or more worklists displayed based on selecting one of the one or more clinical parameters, the one or more non-clinical parameters, or a combination thereof, by the one or more stakeholders. For example, a clinician may sort worklists by the type of disease, such as grouping all cases related to neurological conditions together. This allows the healthcare team to focus on a specific set of cases, improving the efficiency of diagnosis and treatment. Further, if a worklist is sorted by report status, cases that are still pending or in progress can be displayed at the top. This helps ensure that incomplete reports are addressed promptly, avoiding delays in patient care. Further, the user interface unit (210) may allow the one or more worklists to be sorted by urgency. In this case, a high-urgency case like a heart attack or stroke can be moved to the top of the list, ensuring it gets the immediate attention it requires.

Further, the one or more functionalities may correspond to generating alerts, re-ordering the one or more worklists, sharing details with the one or more stakeholders, or a combination thereof. For example, if a radiologist detects a suspicious lesion or tumor in an X-ray or CT scan, the user interface unit (210) may automatically generate an alert to notify the relevant healthcare team members, such as the oncologist or surgeon, prompting immediate action. Further, if the user interface unit (210) detects a rapidly deteriorating condition, such as a heart failure, based on medical images or clinical data, an urgent alert may be sent out to notify all relevant stakeholders of the critical status. This helps prioritize treatment and intervention before the situation worsens.

Further, for example, when a critical case, such as a stroke detected on a CT scan, is flagged by the user interface unit (210), the worklist containing this case may automatically be moved to the top of the list, ensuring that it is addressed first, ahead of less urgent cases. Further, a clinician reviewing a patient's history may notice a significant change in condition or a risk factor, prompting them to manually reorder the worklist. This ensures that high-priority cases receive immediate attention, even if they are not flagged as urgent by the system initially. For example, while sharing the one or more users details with the one or more stakeholders if a radiologist observes an unusual finding in a patient’s CT scan, they may use the user interface unit (210) to securely share the scan results along with any relevant medical history with the neurologist and cardiologist on the same case, ensuring everyone is informed and may collaborate effectively. In one non-limiting embodiment, a physician may want to share a patient’s previous diagnoses or detailed risk factors with other members of the care team. By using the communication interface, the one or more stakeholders may quickly and securely transmit this information to all stakeholders, ensuring a coordinated approach to the patient’s care.

In yet another embodiment, the user interface unit (210) of the application server may be configured to provide the communication interface for collaborative communication between the one or more stakeholders based on the displayed one or more worklists. Further, the communication interface may be provided for the collaborative communication corresponding to each of the one or more users. Further, the collaborative communication between the one or more stakeholders may be performed on the one or more medical images, the one or more pathological features, corresponding to each of the one or more users. Further, the communication interface may be integrated with the one or more worklists to allow the one or more stakeholders to access the one or more medical imaging data and the associated one or more pathological features for each user from the one or more users. This integration enables real-time collaboration and coordination of patient management by providing a unified platform where stakeholders can review and discuss medical images, detect critical conditions, and determine the next steps in the patient’s care plan. Through this interconnected system, healthcare providers may efficiently track patient progress, make informed decisions, and streamline the coordination process, ensuring timely and effective treatment. Further, the collaborative communication may correspond to one of secure messaging, instant messaging, secure document sharing or a combination thereof. Further, the secure messaging may ensure at least one of a HIPPA (Health Insurance Portability and Accountability Act) compliance, GDPR (General Data Protection Regulation) compliance, or other similar established data protection regulations.

In one embodiment, the receiving unit (212) of the application server (104) is disclosed. The receiving unit (210) comprises suitable logic, circuitry, interfaces, and/or code that may be configured for automated patient coordination. Further, the receiving unit (210) may be configured to receive the one or more medical images that may be associated with the one or more users. Further, the one or more medical images may be captured using the image acquisition unit (214).

Additionally, the receiving unit (212) may be configured to handle large volumes of medical imaging data with high efficiency, which is particularly important when dealing with multi-image datasets generated during complex diagnostic procedures. Further, the receiving unit (212) may integrate error-handling and validation mechanisms to ensure that all received medical images are intact and correctly formatted, enabling smooth processing by subsequent system components, such as the image viewing interface and worklist management tools.

In one embodiment, the image acquisition unit (214) of the application server (104) is disclosed. The image acquisition unit (214) comprises suitable logic, circuitry, interfaces, and/or code that may be configured for automated patient coordination. Further, one or more medical images correspond to one of X-rays, Computed Tomography (CT) scans, Magnetic Resonance Imaging (MRI), Ultrasound, Positron Emission Tomography (PET) scans, or a combination thereof. Further, the image acquisition unit (214) may be configured to capture medical images. Further, the one or more medical images may be associated with the image acquisition unit such as, but not limited to, X-ray machines, CT scan machines, MRI machine, PET scan machine or a combination thereof.

Further, the image acquisition unit (214) may automatically detect the one or more pathological features from the one or more medical images. Further, the one or more pathological features may be detected by utilizing one or more deep learning techniques. In an embodiment, the one or more deep learning techniques may correspond to one of a UNet or a UNet++, DeepLab, Fully Convolutional Network (FCN), SegNet, R2U-Net (Recurrent Residual U-Net), ResUNet (U-Net with ResNet backbone), Densely Connected U-Net (DC-UNet), Mask R-CNN, or a combination of the same. Further, the one or more pathological features may correspond to at least one of, but not limited to, infections condition, neurological condition, ontological conditions, or a combination thereof. Further, the image acquisition unit (214) in coordination with the one or more deep learning techniques, may be configured to process the one or more medical images by extracting the meaningful features from the one or more medical images. Further, the meaningful features may correspond to a classification of pathological conditions corresponding to infections, neurological and ontological conditions. Further, the neurological and ontological conditions may correspond to a cavity, bleed, lung nodule, or a combination thereof. Further, the deep learning techniques may be configured to identify and segment the affected anatomical parts and detect the presence of infections, Neurological or and Oncological findings based on key features such as presence of signs of cavity, bleed, mass etc.

In an embodiment, after detecting one or more pathological features utilizing the one or more deep learning techniques, the application server (104) is configured to generate one or more worklists corresponding the one or more users. The one or more worklists are generated based on the one or more pathological features identified by utilizing the one or more deep learning techniques. In an exemplary embodiment, the one or more worklists may be organized such that each worklist corresponds to a single patient. In the same manner, multiple worklists can be created for multiple patients, with each worklist containing the necessary tasks and information specific to that patient’s care. This structure allows for efficient management and tracking of tasks across a diverse group of patients. In another exemplary embodiment, the one or more worklists may be organized in such a way that the one or more worklists are grouped based on medical domain, the identified one or more pathological features belongs to. Accordingly, the one or more worklists may be displayed to the one or more stakeholders based on the one or more pathological features identified from the one or more medical images utilizing the one or more deep learning techniques.

In one embodiment, the integration unit (216) of the application server (104) is disclosed. The user interface unit (210) comprises suitable logic, circuitry, interfaces, and/or code that may be configured for automated patient coordination. Further, the integration unit (216) may be configured to integrate the one or more worklists and the communication interface with an image viewing interface for facilitating viewing of the one or more medical images based on the collaborative communication. Further, the integration unit (216) may integrate the one or more medical images associated with a user from the one or more users, with a clinical information associated with the user to detect classification and quantification associated with the one or more pathological features. Further, the quantification and classification associated with the one or more pathological features may correspond to at least of an Intracranial Haemorrhage, Midline shift, Stroke, Lung Nodule, and a combination thereof. Further, the clinical information may comprise at least one of user history, risk factor, symptoms, typical anatomical changes, age, smoking history, personalized risk profile, clinical guidelines, previous diagnosis notes, family history of disease, or a combination thereof. In another embodiment, the integration unit (216) is configured for integration one of the one or more worklists, the communication interface, the patient coordination report, or a combination thereof with a patient management in a healthcare infrastructure. Further, the integration with the patient workflow management may be configured to facilitate at least one of scheduling, progress tracking, follow-up coordination, or a combination thereof.

In an exemplary embodiment, the integration unit (216) may be configured to analyze longitudinal imaging and clinical data to track disease progression over time. The integration unit may be configured to detect pathologies and classify them using a clinical data model. Further, once the pathologies such as Intracranial Hemorrhage (ICH), Stroke, or Lung Nodules are detected, the integration unit (216) may categorize the one or more pathologies and provide a detailed insights into the diagnosis. Additionally, the integration unit (216) may be configured to generate a comprehensive report that corresponds to critical information like subtype, volume, and other relevant details. Further, the reports may be accessible to clinicians, aiding in informed decision-making and facilitating timely, accurate follow-up care to guide patient management. This integration ensures precise pathology classification and quantification, optimizing patient outcomes.

A person skilled in the art will understand that the scope of the disclosure should not be limited to a specific domain and using the aforementioned techniques. Further, the examples provided in supra are for illustrative purposes and should not be construed to limit the scope of the disclosure.

### Example 01

Let us delve into a detailed working example of the present disclosure.

Example 01: X is a 54-year-old patient who presents with a series of concerning symptoms, including chronic headaches, dizziness, and blurred vision, which have progressively worsened over the past few weeks.

X’s primary care physician, concerned about the possibility of a neurological condition, orders a comprehensive CT scan of the brain to investigate further. The CT images are captured using a high-resolution imaging unit, providing detailed scans that can potentially reveal the underlying cause of X's symptoms. These images are uploaded into the hospital’s central medical imaging system, where they are available for analysis and review by the medical team. Upon receiving the CT images, the system’s processor immediately begins its analysis. Utilizing advanced deep learning techniques, the AI model scans the images for any signs of abnormality or pathology. The AI detects a suspicious area located within X's brain, indicating the potential presence of a bleed. The system flags this finding as critical and places X’s case at the top of the worklist, ensuring it gets immediate attention from the healthcare team. The AI’s detection of this bleed is integrated into the platform’s worklist management system, which then displays the case to relevant stakeholders, including radiologists, neurologists, and surgeons, each with access to the same data.

Meanwhile, another patient, Y, is undergoing a similar workup due to persistent back pain. Y's MRI images are also captured and analyzed using the same system, revealing a possible herniated disk in the lumbar spine. Unlike X, Y’s case is categorized as urgent but not critical, and as such, it is placed further down the worklist for review. While both X and Y are experiencing significant health issues, the system prioritizes X’s case due to the potentially life-threatening nature of the brain bleed. The worklist is automatically updated, adjusting the order of cases based on severity, urgency, and medical history.

As Y’s case is being reviewed by the medical team, an alert is triggered by the system due to the high-risk score detected in X’s case. The system generates an instant notification to all relevant clinicians involved in patient care. This alert notifies the medical professionals that X's brain bleed, identified through the AI detection, requires immediate intervention. The alert is automatically shared with other medical personnel, including the neurosurgeon and neurologist, who are now able to see the urgency of the situation while also reviewing Y’s case. Since the system is fully integrated and accessible to all medical staff, the critical nature of X’s condition is communicated seamlessly, ensuring all team members are on the same page and no time is wasted.

X's worklist contains clinical parameters that include the suspected bleed type, CT findings, and the report status, as well as non-clinical parameters such as urgency, physician preferences, and patient history. The system provides a dynamic filtering and sorting functionality that allows stakeholders to customize the worklist based on specific needs. For example, the neurologist, upon viewing the worklist, can sort by severity or disease type, ensuring that X’s case stays at the top for immediate attention.

As the worklist is being displayed to the healthcare team, the communication interface within the platform becomes critical. X’s case, along with its associated CT findings, is discussed in real-time by the stakeholders. The neurologist and the surgeon engage in secure messaging, exchanging insights and confirming that the suspected bleed requires urgent surgery. The secure chat system embedded within the platform is HIPAA-compliant, ensuring all communications are protected. In parallel, radiologists review the images, and the AI outputs are directly linked to the imaging data, allowing them to confirm the bleed size, location, and characteristics. With a simple click, the medical images and pathology findings are linked to the conversation, making the clinical discussion more focused and actionable.

Y’s case, though urgent, is managed differently. The back pain symptoms, while concerning, are not life-threatening. The orthopedic team and the neurologist assigned to Y’s case communicate using the same platform, though in a less urgent fashion. Y’s MRI images reveal a bulging disc, which, while requiring attention, does not demand immediate intervention. The specialists discuss Y’s situation, reviewing the MRI findings and agreeing to a conservative treatment plan with physical therapy. Y's case is updated in the worklist to reflect the treatment plan, and the corresponding patient coordination report is generated.

In X's case, as the collaborative discussion progresses, the patient coordination report is automatically created by the system, consolidating all findings and communication into a structured document. This report includes the AI-generated tumor detection data, the radiologist’s confirmation of the bleed, the neurologist's assessment of symptoms, and the surgeon`s recommendations for surgery. The report is updated in real-time as stakeholders continue to add their comments or modify findings. Both the neurologist and the surgeon can update the report as they refine their approach, ensuring it is always current and comprehensive.

The integration of AI-based disease detection ensures that all findings are consistently accurate and objective. For example, the AI system quantifies the bleed size, giving clinicians a precise measurement that is included directly in the report. This quantitative data, along with the clinical context (e.g., age, family history of bleed, hypertension etc), provides a holistic view of X’s condition. By integrating medical images and clinical information, the system reduces human error and helps clinicians make well-informed decisions.

Both X and Y’s cases benefit from seamless integration with the hospital’s patient workflow management system. For X, this means that once the surgical team confirms the need for a surgeon, the system helps in scheduling the procedure. All relevant stakeholders are notified, and the patient’s care pathway is tracked efficiently from diagnosis to treatment. Similarly, for Y, the system schedules follow-up appointments for physical therapy and tracks progress, ensuring that no steps in the care process are missed. Throughout this process, the communication interface continues to serve as a key tool for cross-specialty collaboration. For X, the platform allows real-time communication between radiologists, neurologists, and surgeon, ensuring that all necessary specialists are involved in the decision-making process. The built-in messaging system is encrypted and compliant with healthcare regulations, ensuring secure and efficient exchanges of information. Furthermore, patient information, including medical images, reports, and notes, are organized per patient, ensuring that no information is lost or overlooked.

In conclusion, the integration of medical images, AI-based disease detection, worklist management, and secure communication interfaces in a single platform ensures that both X and Y’s cases are handled efficiently and accurately. X, with a critical neurological condition, benefits from fast-tracked diagnosis and treatment planning, while Y’s condition is managed with appropriate urgency. The automated system allows healthcare professionals to collaborate seamlessly, update reports in real-time, and ensure that all necessary steps in the care process are followed, resulting in improved patient outcomes for both individuals. The alert about X’s high-risk condition, shared in real-time with all relevant clinicians, underscores the value of the platform's integration, ensuring timely and appropriate interventions.

A person skilled in the art will understand that the scope of the disclosure is not limited to scenarios based on the aforementioned factors and using the aforementioned techniques, and that the examples provided do not limit the scope of the disclosure.

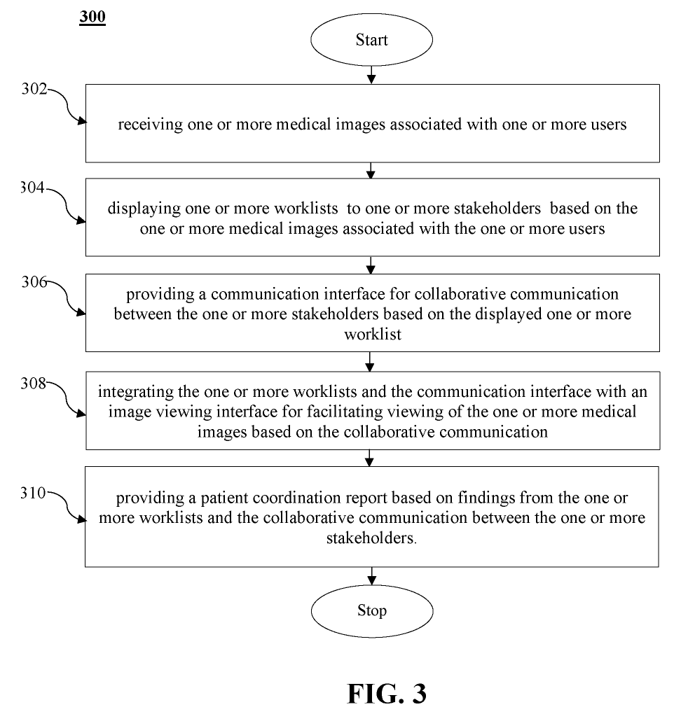

Referring to FIG. 3, a flowchart that illustrates a method (300) for automated patient coordination, in accordance with at least one embodiment of the present subject matter. The method (300) may be implemented by the application server (104) including the processor (202) and the memory (204) communicatively coupled to the processor (202) and the memory (204) is configured to store processor-executable programmed instructions, caused the processor to perform the following steps.

At step (302), the processor (202) is configured to receive one or more medical images associated with one or more users. Further, at step (304) the processor (202) is configured to display one or more worklists to one or more stakeholders based on the one or more medical images associated with the one or more users. Further, at step (306) the processor (202) is configured to provide a communication interface for collaborative communication between the one or more stakeholders based on the displayed one or more worklists. Further, at step (308) the processor (202) is configured to integrate the one or more worklists and the communication interface with an image viewing interface for facilitating viewing of the one or more medical images based on the collaborative communication. Further, at step (310) the processor (202) is configured to provide a patient coordination report based on findings from the one or more worklists and the collaborative communication between the one or more stakeholders.

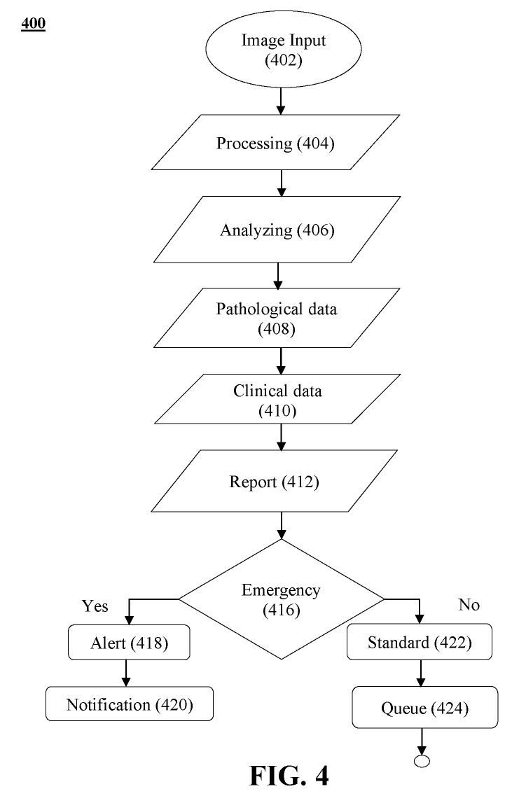

FIG. 4 illustrates a flow diagram (400) of automated patient coordination, in accordance with an exemplary embodiment of the present subject matter. The process begins with step (402) by inputting the one or more medical images input (402). Further, at step (404) the one or more medical images may be processed to enhance the quality and extract the one or more relevant features that may be related to patients’ health conditions. Further, at step (406), examination of the one or more patterns or abnormalities are analyzed. Further, at step (408) the extracted information may be categorized into the pathological data (408). Further, at step (410) the stored clinical data of the patient may be used. For example, the clinical data may correspond to a medical history, previous radiology report or a symptom related to diagnosis, or a combination thereof. Further, at step (412) the summarized report of each patient may be generated. Further, at step (416) the system (100) may provide the alert (418) based on the medical emergency of the user. Further, in case of emergency, the system (100) may be configured to notify the one or more stakeholders as a notification (420) to ensure prompt answer. At step (422), if the case is not an emergency, a standard (422) procedure is followed, where it is placed in a queue (424) for routine processing.

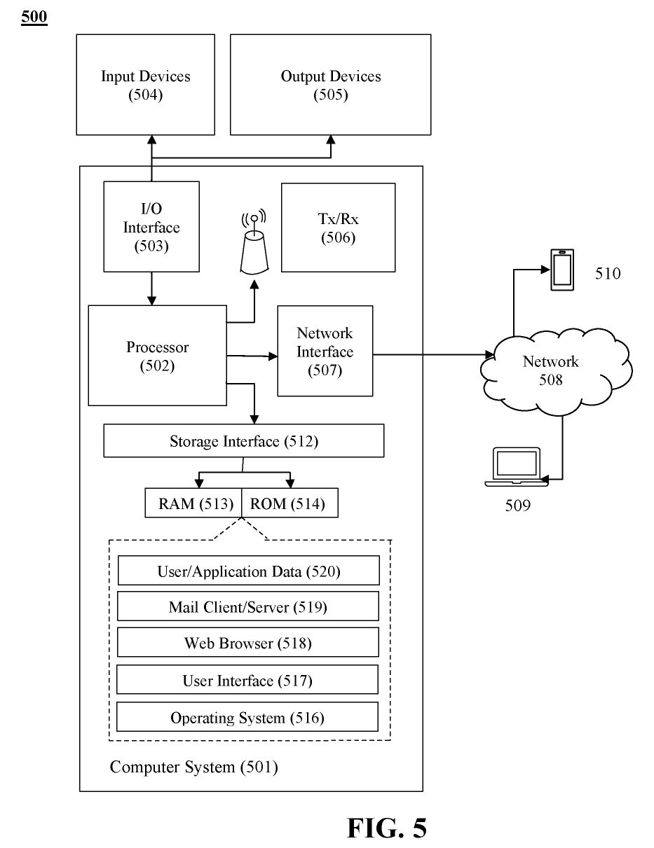

FIG. 5 illustrates a block diagram of an exemplary computer system (501) for implementing embodiments consistent with the present disclosure.

Variations of computer system (501) may be used for the automated patient coordination. The computer system (501) may comprise a central processing unit (“CPU” or “processor”) (502). The processor (502) may comprise at least one data processor for executing program components for executing user or system generated requests. A user may include a person, a person using a device such as those included in this disclosure, or such a device itself. Additionally, the processor (502) may include specialized processing units such as integrated system (bus) controllers, memory management control units, floating point units, graphics processing units, digital signal processing units, or the like. In various implementations the processor (502) may include a microprocessor, such as AMD Athlon, Duron or Opteron, ARM’s application, embedded or secure processors, IBM PowerPC, Intel’s Core, Itanium, Xeon, Celeron or other line of processors, for example. Accordingly, the processor (502) may be implemented using mainframe, distributed processor, multi-core, parallel, grid, or other architectures. Some embodiments may utilize embedded technologies like application-specific integrated circuits (ASICs), digital signal processors (DSPs), or Field Programmable Gate Arrays (FPGAs), for example.

Processor (502) may be disposed in communication with one or more input/output (I/O) devices via I/O interface (503). Accordingly, the I/O interface (503) may employ communication protocols/methods such as, without limitation, audio, analog, digital, monoaural, RCA, stereo, IEEE-1394, serial bus, universal serial bus (USB), infrared, PS/2, BNC, coaxial, component, composite, digital visual interface (DVI), high-definition multimedia interface (HDMI), RF antennas, S-Video, VGA, IEEE 802.n/b/g/n/x, Bluetooth, cellular (e.g., code-division multiple access (CDMA), high-speed packet access (HSPA+), global system for mobile communications (GSM), long-term evolution (LTE), WiMAX, or the like, for example.

Using the I/O interface (503), the computer system (501) may communicate with one or more I/O devices. For example, the input device (504) may be an antenna, keyboard, mouse, joystick, (infrared) remote control, camera, card reader, fax machine, dongle, biometric reader, microphone, touch screen, touchpad, trackball, sensor (e.g., accelerometer, light sensor, GPS, gyroscope, proximity sensor, or the like), stylus, scanner, storage device, transceiver, video device/source, or visors, for example. Likewise, an output device (505) may be a user’s smartphone, tablet, cell phone, laptop, printer, computer desktop, fax machine, video display (e.g., cathode ray tube (CRT), liquid crystal display (LCD), light- emitting diode (LED), plasma, or the like), or audio speaker, for example. In some embodiments, a transceiver (506) may be disposed in connection with the processor (502). The transceiver (506) may facilitate various types of wireless transmission or reception. For example, the transceiver (506) may include an antenna operatively connected to a transceiver chip (example devices include the Texas Instruments® WiLink WL1283, Broadcom® BCM4750IUB8, Infineon Technologies® X-Gold 618-PMB9800, or the like), providing IEEE 802.11a/b/g/n, Bluetooth, FM, global positioning system (GPS), and/or 2G/3G/5G/6G HSDPA/HSUPA communications, for example.

In some embodiments, the processor (502) may be disposed in communication with a communication network (508) via a network interface (507). The network interface (507) is adapted to communicate with the communication network (508). The network interface (507) may employ connection protocols including, without limitation, direct connect, Ethernet (e.g., twisted pair 10/100/1000 Base T), transmission control protocol/internet protocol (TCP/IP), token ring, or IEEE 802.11a/b/g/n/x, for example. The communication network (508) may include, without limitation, a direct interconnection, local area network (LAN), wide area network (WAN), wireless network (e.g., using Wireless Application Protocol), or the Internet, for example. Using the network interface (507) and the communication network (508), the computer system (501) may communicate with devices such as shown as a laptop (509) or a mobile/cellular phone (510). Other exemplary devices may include, without limitation, personal computer(s), server(s), fax machines, printers, scanners, various mobile devices such as cellular telephones, smartphones (e.g., Apple iPhone, Blackberry, Android-based phones, etc.), tablet computers, desktop computers, eBook readers (Amazon Kindle, Nook, etc.), laptop computers, notebooks, gaming consoles (Microsoft Xbox, Nintendo DS, Sony PlayStation, etc.), or the like. In some embodiments, the computer system (501) may itself embody one or more of these devices.

In some embodiments, the processor (502) may be disposed in communication with one or more memory devices (e.g., RAM 513, ROM 514, etc.) via a storage interface (512). The storage interface (512) may connect to memory devices including, without limitation, memory drives, removable disc drives, etc., employing connection protocols such as serial advanced technology attachment (SATA), integrated drive electronics (IDE), IEEE-1394, universal serial bus (USB), fiber channel, small computer systems interface (SCSI), etc. The memory drives may further include a drum, magnetic disc drive, magneto-optical drive, optical drive, redundant array of independent discs (RAID), solid-state memory devices, or solid-state drives, for example.

The memory devices may store a collection of program or database components, including, without limitation, an operating system (516), user interface application (517), web browser (518), mail client/server (519), user/application data (520) (e.g., any data variables or data records discussed in this disclosure) for example. The operating system (516) may facilitate resource management and operation of the computer system (501). Examples of operating systems include, without limitation, Apple Macintosh OS X, UNIX, Unix-like system distributions (e.g., Berkeley Software Distribution (BSD), FreeBSD, NetBSD, OpenBSD, etc.), Linux distributions (e.g., Red Hat, Ubuntu, Kubuntu, etc.), IBM OS/2, Microsoft Windows (XP, Vista/7/8, etc.), Apple iOS, Google Android, Blackberry OS, or the like.

The user interface (517) is for facilitating the display, execution, interaction, manipulation, or operation of program components through textual or graphical facilities. For example, user interfaces (517) may provide computer interaction interface elements on a display system operatively connected to the computer system (501), such as cursors, icons, check boxes, menus, scrollers, windows, or widgets, for example. Graphical user interfaces (GUIs) may be employed, including, without limitation, Apple Macintosh operating systems’ Aqua, IBM OS/2, Microsoft Windows (e.g., Aero, Metro, etc.), Unix X-Windows, or web interface libraries (e.g., ActiveX, Java, JavaScript, AJAX, HTML, Adobe Flash, etc.), for example.

In some embodiments, the computer system (501) may implement a web browser (518) stored program component. The web browser (518) may be a hypertext viewing application, such as Microsoft Internet Explorer, Google Chrome, Mozilla Firefox, Apple Safari, or Microsoft Edge, for example. Secure web browsing may be provided using HTTPS (secure hypertext transport protocol), secure sockets layer (SSL), Transport Layer Security (TLS), or the like. Web browsers may utilize facilities such as AJAX, DHTML, Adobe Flash, JavaScript, Java, or application programming interfaces (APIs), for example. In some embodiments the computer system (501) may implement a mail client/server (519) stored program component. The mail server (519) may be an Internet mail server such as Microsoft Exchange, or the like. The mail server may utilize facilities such as ASP, ActiveX, ANSI C++/C#, Microsoft .NET, CGI scripts, Java, JavaScript, PERL, PHP, Python, or WebObjects, for example. The mail server (519) may utilize communication protocols such as internet message access protocol (IMAP), messaging application programming interface (MAPI), Microsoft Exchange, post office protocol (POP), simple mail transfer protocol (SMTP), or the like. In some embodiments, the computer system (501) may implement a mail client (520) stored program component. The mail client (520) may be a mail viewing application, such as Apple Mail, Microsoft Entourage, Microsoft Outlook, or Mozilla Thunderbird.

In some embodiments, the computer system (501) may store user/application data (521), such as the data, variables, records, or the like as described in this disclosure. Such databases may be implemented as fault-tolerant, relational, scalable, secure databases such as Oracle or Sybase, for example. Alternatively, such databases may be implemented using standardized data structures, such as an array, hash, linked list, struct, structured text file (e.g., XML), table, or as object-oriented databases (e.g., using ObjectStore, Poet, Zope, etc.). Such databases may be consolidated or distributed, sometimes among the various computer systems discussed above in this disclosure. It is to be understood that the structure and operation of any computer or database component may be combined, consolidated, or distributed in any working combination.

Furthermore, one or more computer-readable storage media may be utilized in implementing embodiments consistent with the present invention. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer readable medium” should be understood to include tangible items and exclude carrier waves and transient signals, i.e., non-transitory. Examples include Random Access Memory (RAM), Read- Only Memory (ROM), volatile memory, non-volatile memory, hard drives, Compact Disc (CD) ROMs, Digital Video Disc (DVDs), flash drives, disks, and any other known physical storage media.

The present disclosure addresses the inefficiencies and limitations of conventional patient coordination systems, which often lack seamless integration between medical imaging, worklist management, and stakeholders. Traditional systems typically operate in silos, resulting in fragmented workflows, delays in decision-making, and inefficient resource allocation. The disclosed invention introduces an automated patient coordination method that integrates medical image analysis, collaborative communication, and dynamic worklist management into a single, streamlined workflow. The system begins by receiving medical images associated with patients and processing them to generate worklists tailored to different stakeholders, such as radiologists, physicians, and care coordinators. These worklists prioritize cases based on urgency and relevance, ensuring that critical findings receive immediate attention. A communication interface is provided, enabling real-time collaboration between stakeholders directly within the workflow. This integration allows healthcare professionals to discuss cases, share insights, and make informed decisions without switching between multiple systems. Additionally, the worklists and communication interface are seamlessly integrated with an image viewing interface, facilitating the direct examination of medical images alongside discussions and annotations. This comprehensive approach enhances efficiency, reduces delays, and ensures that relevant medical findings are accurately documented. Finally, the system generates a patient coordination report that consolidates findings from the worklists and collaborative communications, ensuring that all relevant information is available for follow-ups and treatment planning. By dynamically integrating medical imaging, communication, and patient coordination, this invention overcomes the limitations of traditional systems and significantly improves healthcare delivery.

By continuously monitoring and adjusting provider selection in real-time based on performance metrics, the system ensures optimal resource utilization while preventing bottlenecks and service overloads. This dynamic recalibration process allows for a self-sustaining and scalable solution that efficiently adapts to growing demand. By eliminating the need for manual intervention, the system significantly reduces operational overhead and enhances overall reliability. Leveraging real-time performance data, automated retries, and predictive analytics driven by machine learning, the system intelligently optimizes provider selection, ensuring seamless, efficient, and high-quality communication experiences. This adaptive approach enhances system resilience and ensures consistent performance across varying workloads.

Various embodiments of the disclosure encompass numerous advantages including methods and systems for the automated patient coordination. The disclosed method and system have several technical advantages, but not limited to the following:
Automated and Efficient Patient Coordination: The method automates patient coordination by integrating medical images, worklists, and communication interfaces, improving workflow efficiency. Stakeholders are able to collaborate seamlessly, enhancing care coordination and reducing errors or delays in decision-making.
AI-Based Disease Detection: The method integrates AI-powered disease detection within the platform, automatically analyzing medical images and embedding findings directly within the imaging viewer (e.g., PACS-like system). This enables real-time review without the need to switch between platforms. AI findings, along with clinical data such as lab results, patient history, and prior imaging, provide context-aware insights and are automatically included in reports, eliminating manual entry errors.
Enhanced Collaboration and Communication: The method offers a secure communication interface for real-time collaboration between stakeholders. Clinicians can share medical images, pathological findings, and case details through secure chat and document sharing, ensuring compliance with HIPAA and GDPR regulations. Messages are organized per patient case, making the conversation more patient-centric and actionable.
Real-Time Worklist Prioritization: The method uses AI-driven prioritization to automatically move critical cases, such as acute strokes or hemorrhages, to the top of the worklist. This feature ensures that high-priority cases are quickly addressed, reducing response times and improving patient outcomes. Additionally, the system provides customizable filtering options, allowing stakeholders to sort cases by disease type, urgency, or physician preference.
Comprehensive Integration with Medical Data: The system integrates medical images, clinical data, and AI insights to provide a holistic view of the patient’s condition. This enables more accurate diagnoses and supports personalized treatment plans. AI outputs are embedded in reports, and quantitative analysis linked to disease findings ensures comprehensive and structured reporting.
Quantification and Reporting: Automated report generation leverages AI findings to create structured, standardized reports. The system also includes quantitative analysis, linking measurement data to disease findings for more detailed reporting. Seamless integration with Electronic Health Records (EHR) nd PACS systems ensures reports are automatically pushed to the patient’s medical record, streamlining the workflow and reducing manual entry.
Cross-Specialty Coordination: The method allows cross-specialty collaboration by providing a single worklist accessible to radiologists, ER physicians, and referring clinicians. This ensures faster coordination, with all relevant stakeholders having access to the same worklist. This integration accelerates decision-making and care delivery, especially in critical situations.
Emergency Alerts & Notifications: The system provides instant alerts for AI-detected critical findings, notifying the relevant healthcare professionals in real-time. Customizable alert settings allow notifications to be configured based on severity, specialty, or workflow preferences, ensuring timely responses to urgent cases.
Continuous Updates and Findings Tracking: The method allows stakeholders to continuously update findings in the patient coordination report. These real-time updates ensure that all parties have access to the latest information, improving the coordination of care.
Seamless Integration with Healthcare Workflow: By integrating the patient coordination system with existing healthcare workflow management tools, the method enhances scheduling, progress tracking, and follow-up coordination. This reduces administrative overhead and ensures a more efficient care process.
Secure and Efficient Information Sharing: The built-in secure chat system within the platform allows clinicians to share medical images, reports, and findings in real-time, ensuring that communication is both secure and efficient. This supports better decision-making by providing stakeholders with all relevant information in one location.
Scalable and Adaptable to Various Medical Conditions: The method is designed to handle diverse medical conditions, from infectious to oncological diseases. Its scalable architecture makes it adaptable to different specialties and care settings, ensuring that it can meet the needs of a wide range of healthcare providers.

In summary, the method for automated patient coordination addresses the limitations of traditional patient coordination systems by enhancing collaboration and streamlining communication among stakeholders. Traditional systems often rely on manual processes, resulting in inefficiencies, delayed decision-making, and a lack of integrated communication. In contrast, the claimed method leverages a processor to receive medical images associated with patients and display relevant worklists to stakeholders, enabling more efficient management of patient data. The system provides a communication interface that facilitates real-time collaborative communication among stakeholders based on the displayed worklists, improving coordination. Additionally, the integration of worklists and the communication interface with an image viewing platform enhances the viewing and analysis of medical images, promoting better decision-making. By providing a comprehensive patient coordination report based on findings from the worklists and collaborative communication, the system improves the efficiency of patient management, reduces manual intervention, and enhances overall healthcare delivery by ensuring accurate and timely coordination among stakeholders.

The claimed invention of the method and the system for automated patient coordination provider involves tangible components, processes, and functionalities that interact to achieve specific technical outcomes. The system integrates various elements such as processors, memory, databases, weights, and other techniques for automated patient coordination.

The present disclosure introduces an automated patient coordination system that significantly enhances the efficiency and reliability of healthcare workflows. By leveraging medical images associated with users, the system dynamically generates worklists for stakeholders, allowing for efficient and streamlined communication. The system enables collaborative communication between stakeholders through an integrated communication interface, which is tied to the worklist and medical image viewing interface. This allows users to engage in real-time discussions and make informed decisions based on medical findings.. As a result, the system ensures timely and accurate information exchange, minimizing delays and optimizing patient care delivery while maintaining a high level of adaptability and responsiveness.

In summary, the present disclosure addresses the inefficiencies of traditional patient coordination systems, which often rely on manual intervention and lack real-time adaptability. Traditional systems can experience delays, miscommunications, and errors that negatively affect patient care. In contrast, the disclosed system utilizes automation and integrated tools, such as dynamic worklist management and collaborative communication interfaces, to streamline the coordination process. By leveraging real-time feedback and continuously optimizing stakeholder interactions, the system enhances the quality and timeliness of patient care, reduces delays, and ensures a more effective coordination process. Ultimately, this approach leads to improved patient outcomes through more efficient and responsive management of medical workflows.

In light of the above-mentioned advantages and the technical advancements provided by the disclosed method and system, the claimed steps as discussed above are not routine, conventional, or well understood in the art, as the claimed steps enable the following solutions to the existing problems in conventional technologies. Further, the claimed steps clearly bring an improvement in the functioning of the device itself as the claimed steps provide a technical solution to a technical problem.

The present disclosure may be realized in hardware, or a combination of hardware and software. The present disclosure may be realized in a centralized fashion, in at least one computer system, or in a distributed fashion, where different elements may be spread across several interconnected computer systems. A computer system or other apparatus adapted for carrying out the methods described herein may be suited. A combination of hardware and software may be a general-purpose computer system with a computer program that, when loaded and executed, may control the computer system such that it carries out the methods described herein. The present disclosure may be realized in hardware that comprises a portion of an integrated circuit that also performs other functions.

A person with ordinary skills in the art will appreciate that the systems, modules, and sub-modules have been illustrated and explained to serve as examples and should not be considered limiting in any manner. It will be further appreciated that the variants of the above disclosed system elements, modules, and other features and functions, or alternatives thereof, may be combined to create other different systems or applications.

Those skilled in the art will appreciate that any of the aforementioned steps and/or system modules may be suitably replaced, reordered, or removed, and additional steps and/or system modules may be inserted, depending on the needs of a particular application. In addition, the systems of the aforementioned embodiments may be implemented using a wide variety of suitable processes and system modules, and are not limited to any particular computer hardware, software, middleware, firmware, microcode, and the like. The claims can encompass embodiments for hardware and software, or a combination thereof.

While the present disclosure has been described with reference to certain embodiments, it will be understood by those skilled in the art that various changes may be made, and equivalents may be substituted without departing from the scope of the present disclosure. In addition, many modifications may be made to adapt a particular situation or material to the teachings of the present disclosure without departing from its scope. Therefore, it is intended that the present disclosure is not limited to the particular embodiment disclosed, but that the present disclosure will include all embodiments falling within the scope of the appended claims.

We claim:

1. A method (300) for automated patient coordination, wherein the method (300) comprises:
receiving (302), by a processor (202), one or more medical images associated with one or more users;
displaying (304), by the processor (202), one or more worklists to one or more stakeholders based on the one or more medical images associated with the one or more users;
providing (306), by the processor (202), a communication interface for collaborative communication between the one or more stakeholders based on the displayed one or more worklists;
integrating (308), by the processor (202), the one or more worklists and the communication interface with an image viewing interface for facilitating viewing of the one or more medical images based on the collaborative communication; and
providing (310), by the processor (202), a patient coordination report based on findings from the one or more worklists and the collaborative communication between the one or more stakeholders.

2. The method (300) as claimed in claim 1, wherein the one or more medical images being captured using one or more image acquisition units; wherein the one or more medical images correspond to one of X-rays, Computed Tomography (CT) scans, Magnetic Resonance Imaging (MRI), Ultrasound, Positron Emission Tomography (PET) scans, or a combination thereof.

3. The method (300) as claimed in claim 1, comprises automatically detecting one or more pathological features from the one or more medical images,
wherein the one or more pathological features being detected by utilizing one or more deep learning techniques wherein the one or more deep learning techniques corresponds to one of UNet or a UNet++, DeepLab, Fully Convolutional Network (FCN), SegNet, R2U-Net (Recurrent Residual U-Net), ResUNet (U-Net with ResNet backbone), Densely Connected U-Net (DC-UNet), Mask R-CNN, or a combination thereof;
wherein the one or more pathological features corresponds to at least one of infectious condition, neurological condition, oncological conditions or a combination thereof.

4. The method (300) as claimed in claim 3,
wherein displaying (304) the one or more worklists is performed based on the detection of the one or more pathological features from the one or more medical images;
wherein the each of the one or more worklists comprises at least one or more clinical parameters, one or more non-clinical parameters, or a combination thereof;
wherein the one or more clinical parameters comprises at least one of the one or more pathological features, type of disease, report status, finding present, finding absent or a combination thereof;
wherein the one or more non-clinical parameters comprises at least one of report status, urgency, physician preference, dates, sites, scan status, action pending, modalities, patient demographics or a combination thereof.

5. The method (300) as claimed in claim 4, comprises providing one or more functionalities on the one or more worklists based on one of the one or more clinical parameters, the one or more non-clinical parameters, or a combination thereof;
wherein providing the one or more functionalities corresponds to one of filtering the one or more worklists displayed based on selecting one of the one or more clinical parameters, the one or more non-clinical parameters, or a combination thereof, by the one or more stakeholders, sorting the one or more worklists displayed based on selecting one of the one or more clinical parameters, the one or more non-clinical parameters, or a combination thereof, by the one or more stakeholders, generating alerts, re-ordering the one or more worklists, sharing details with the one or more stakeholders, or a combination thereof.

6. The method (300) as claimed in claim 3, comprises automatically generating a real-time notification alert based on detecting the one or more pathological features belonging to one or more critical conditions, wherein a worklist containing the one or more pathological features belonging to the one or more critical conditions, is automatically move to top of the one or more worklists displayed to the one or more stakeholders.

7. The method (300) as claimed in claim 3, wherein the one or more pathological features in the one or more worklists is updated by the one or more stakeholders.

8. The method (300) as claimed in claim 3, comprises integrating the one or more medical images associated with a user, from the one or more users, with a clinical information associated with the user to detect classification and quantification associated with the one or more pathological features;
wherein the clinical information comprises at least one of user history, risk factor, symptoms, typical anatomical changes, age, smoking history, personalized risk profile, clinical guidelines, previous diagnosis notes, family history of disease, or a combination thereof.

9. The method (300) as claimed in claim 1, wherein the communication interface is provided for the collaborative communication corresponding to each of the one or more users;
wherein the collaborative communication between the one or more stakeholders is performed on the one or more medical images, the one or more pathological features, corresponding to each of the one or more users;
wherein the collaborative communication corresponds to one of secure messaging, instant messaging, secure document sharing or a combination thereof.

10. The method (300) as claimed in claim 1,
wherein the patient coordination report is displayed on the image viewing interface;
wherein the patient coordination report is generated by consolidating the findings from the one or more worklists and the collaborative communication between the one or more stakeholders;
wherein the findings in the patient coordination report is updated by the one or more stakeholders.

11. The method (300) as claimed in claim 1, comprises integrating one of the one or worklists, the communication interface, the patient coordination report, or a combination thereof, with patient workflow management in a healthcare infrastructure; wherein integrating with the patient workflow management facilitates at least one of scheduling, progress tracking, follow-up coordination, or a combination thereof.

12. A system (100) of automated patient coordination, the system (100) comprises:
a processor (202), and
a memory (204) communicatively coupled with the processor (202), wherein the memory (204) is configured to store one or more executable instructions, which cause the processor (202) for:
receiving (302) one or more medical images associated with one or more users;
displaying (304) one or more worklists to one or more stakeholders based on the one or more medical images associated with the one or more users;
providing (306) a communication interface for collaborative communication between the one or more stakeholders based on the displayed one or more worklists;
integrating (308) the one or more worklists and the communication interface with an image viewing interface for facilitating viewing of the one or more medical images based on the collaborative communication; and
providing (310)a patient coordination report based on findings from the one or more worklists and the collaborative communication between the one or more stakeholders.

13. A non-transitory computer-readable storage medium having stored thereon, a set of computer-executable instructions causing a computer comprising one or more processors to perform steps comprising:
receiving. (302) one or more medical images associated with one or more users;
displaying (304) one or more worklists to one or more stakeholders based on the one or more medical images associated with the one or more users;
providing (306) a communication interface for collaborative communication between the one or more stakeholders based on the displayed one or more worklists;
integrating (308) the one or more worklists and the communication interface with an image viewing interface for facilitating viewing of the one or more medical images based on the collaborative communication; and
providing (310) a patient coordination report based on findings from the one or more worklists and the collaborative communication between the one or more stakeholders.
